(19) United States
(12) Patent Application Publication
(10) Pub. No.: US 2009/0305371 A1
(43) Pub. Date: Dec. 10, 2009

(54) METHOD USING INFRARED LIGHT FOR ETHANOL PRODUCTION

(76) Inventors: Lucia Atehortua, Medellin (CO); Mabel Beatriz Esquivia Mercado, Medellin (CO)

Correspondence Address: JOHN J. MARTINEZ MD. JD., 10 PALMER AVENUE, CROTON ON HUDSON, NY, 10520, US

(21) Appl. No.: 12/135,175

(22) Filed: Jun. 7, 2008

(51) Int. Cl. C12P 7/06

(52) U.S. Cl. 435/161

(57) ABSTRACT

The present invention provides a method to optimize the fermentation culture of ethanol wherein said method comprises a device that allows enough illumination of the fermentation culture with a source or sources of light that emit a precise light wavelength with low power energy consumption and low heat generation. In addition, the present invention provides said method where the source or sources of light, if necessary, can be switched on and off, or, when said sources of light have different light wavelengths, they can be switched on an off in alternated cycles.

### FIELD OF THE INVENTION

The present invention relates to a method to improve fermentation of ethanol by the use of a device that optimizes illumination of a yeast fermentation culture. In previous pending unpublished U.S. patent application Ser. No. 11/635,986, which is incorporated herein in its entirety by reference, the first inventor of the present application describes a different purpose method that uses a similar device.

### DESCRIPTION OF PRIOR ART

Growing microorganisms has been described to be affected by the degree of exposure to darkness or to specific light wavelength.

By way of example, U.S. Pat. No. 5,578,472 to Ueda describes a method for the production of ethanol involving microalgae growth under darkness. U.S. Pat. No. 6,558,943 to Li discloses a method for propagating fungi that comprises 30% light and 70% dark cycles.

Specifically growing microorganisms for the production of ethanol by fermentation with yeast, U.S. patent application Ser. No. 11/439,726 by Lee describes a method for yeast fermentation of ethanol that involves the use of minerals which irradiate infrared light wavelengths.

However, there is no description in the prior art of methods with an illuminating system or device that can be flexibly adapted to dark-light cycles or one light wavelength to another light wavelength cycles, in which a light source or light sources emit controlled precise light wavelength with low power energy consumption a low heat generation.

### SUMMARY OF THE INVENTION

The present invention provides a method to optimize the fermentation culture of ethanol wherein said method comprises a device that allows enough illumination of the fermentation culture with a source or sources of light that emit a precise light wavelength with low power energy consumption and low heat generation. In addition, the present invention provides said method where the source or sources of light, if necessary, can be switched on and off, or, when said sources of light have different light wavelengths, they can be switched on an off in alternated cycles.

Specifically, the present invention provides a method for optimizing ethanol fermentation, wherein said method comprises:
A. Inoculating yeast into culture medium in at least one container, wherein the culture medium has a fermentable carbon source, and wherein the container has completely transparent walls;
B. Putting the container inside a tridimensional closed system, wherein the tridimensional closed system has all internal surfaces with all the characteristics of a mirror;
C. Fitting the tridimensional closed system with a least one source of light, wherein the source of light is a light emitting diode (LED);
D. Incubating for at least one determined period of time;

wherein after incubating, ethanol is produced, and wherein the produced ethanol is separated from the culture medium.

In one aspect of the method of the present invention, the yeast is a strain of Saccharomyces cereviseae.

In one embodiment of the method of the present invention, the fermentable carbon source is a saccharide starch.

In another embodiment of the method of the present invention, the fermentable carbon source is a saccharide sugar.

In one more aspect of the method of the present invention, the tridimensional closed system internal surfaces are mirrors.

In one further aspect of the method of the present invention, the LED is selected from the group consisting of white light emitting LED, ultraviolet light emitting LED, violet light emitting LED, blue light emitting LED, green light emitting LED, yellow light emitting LED, orange light emitting LED, red light emitting LED, and infrared light emitting LED.

Moreover, the method of the present invention comprises incubating the container inside de closed tridimensional system with a LED for a determined period of time and incubating the container for an alternating period of time without any light.

Objectives and additional advantages of the present invention will become more evident in the description of the figures, the detailed description of the invention and the claims.

### BRIEF DESCRIPTION OF THE DRAWINGS

FIG. 1. is an illustrative view of a preferred embodiment of the tridimensional closed system of the present invention.

FIG. 2. is another view of the preferred embodiment of the tridimensional closed system of the present invention, in which one of the walls of the tridimensional enclosure is hypothetically open to illustrate that all the internal surfaces (shaded areas) of the enclosure have all the characteristics of mirrors.

### DETAILED DESCRIPTION OF THE INVENTION

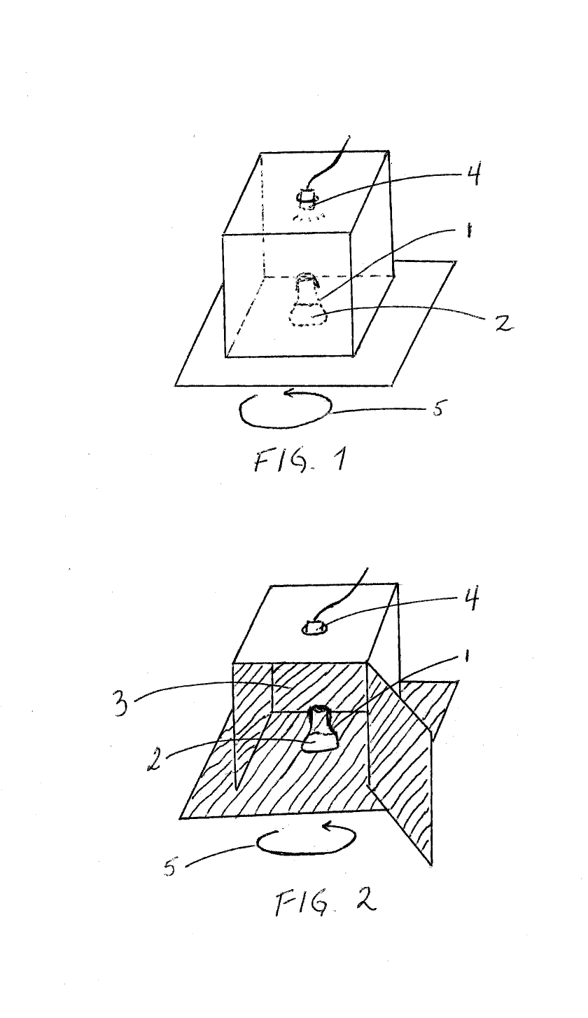

The present invention provides a method for optimizing ethanol fermentation, wherein said method comprises:
A. Inoculating yeast into culture medium in at least one container (1) (FIGS. 1 and 2), wherein the culture medium (2) has a fermentable carbon source, and wherein the container (1) has completely transparent walls;
B. Putting the container (1) inside a tridimensional closed system, wherein the tridimensional closed system has all internal surfaces (3) (shaded areas of FIG. 2) with all the characteristics of a mirror;
C. Fitting the tridimensional closed system with a least one source of light (4), wherein the source of light (4) is a light emitting diode (LED);
D. Incubating for at least one determined period of time;

wherein after incubating, ethanol is produced, and wherein the produced ethanol is separated from the culture medium.

Preferably ethanol is recovered from the culture medium by membrane separation, or ternary azeotropic distillation, or other ethanol recovery methods known in the art.

The definition of the tridimensional closed system of the present invention, in which all the internal walls of said system have all the characteristics of a mirror, includes all kind of enclosures with room or space, (e.g., incubators, bioreactors) to house any type of container for culturing cells.

The tridimensional closed system of the present invention may also have an attached mechanism to agitate the container or containers with transparent walls. Preferably, the container is incubated under constant agitation (5).

In one preferred aspect of the method of the present invention, the yeast is a strain of Saccharomyces cereviseae. More preferably the strain of Saccharomyces cereviseae is the EthanolRed™ from the Fermentis Company, France.

In a preferred embodiment of the present invention the container with transparent walls is an Erlenmeyer flask (1). However, the container of the present invention can also have any geometrical form, as long as, it has transparent walls, in order to allow the penetration of light inside of said container and therefore direct exposure of the culture medium being incubated to the light.

In one preferred embodiment of the method of the present invention, the fermentable carbon source is a saccharide starch. Preferably the starch can be derived from potato powder. The starch can also be derived from corn or other starch sources known in the art.

In another embodiment of the method of the present invention, the fermentable carbon source is a saccharide sugar.

In one more preferred aspect of the method of the present invention, the tridimensional closed system internal surfaces are mirrors.

In one further aspect of the method of the present invention, the LED is selected from the group consisting of white light emitting LED, ultraviolet light emitting LED, violet light emitting LED, blue light emitting LED, green light emitting LED, yellow light emitting LED, orange light emitting LED, red light emitting LED, and infrared light emitting LED.

In a preferred embodiment of the present invention the LED is a infrared light emitting LED, more preferably with a 850 nm wavelength and incubating under the infrared LED is for a determined period of 36 hours.

Moreover, In another embodiment of the method of the present invention comprises incubating the container inside de closed tridimensional system with a LED for a determined period of time and incubating the container for an alternating period of time without any light.

While the description presents the preferred embodiments of the present invention, additional changes can be made in the form and disposition of the parts without distancing from the basic ideas and principles comprised in the claims.

### EXAMPLES

A yeast strain of EthanolRed™ Saccharomyces cereviseae from the Fermentis Company, France was inoculated in a liquid culture medium containing potato starch (250 g/l) in an Erlenmeyer flask. The Erlenmeyer flask was put inside a sealed cubical box wherein the box internal surfaces were mirrors, and where each side of the box measured 55 cm. A central perforation was made on the superior wall of the box in order to install different LEDs. The Erlenmeyer flask with the yeast and the culture medium was incubated under constant agitation at room temperature (25° C.) for up to 60 hours each time in a box equipped with a LED light emitting a total of 2.5 Watts, or no LED. The LEDs used were a white LED, a Blue LED, a Yellow LED, a red LED, an infrared LED, and no LED (darkness). The white, blue, yellow and red LEDs were commercially available 2.5 Watts LED lamps. The Infrared LED was built with 25 commercially available 850 nm 100 mW 5 mm bulb lights.

The experiment was repeated at least three times for each LED, and under darkness.

Samples of the culture medium for each LED, and under darkness were taken each 12 hour interval, 12 hours (T1), 24 hours (T2), 36 hours (T3), 48 hours (T4), and 60 hours (T5). The samples were analyzed for ethanol (% V/V) using High Performance Liquid Chromatography (HPLC).

Temperature was monitored and illumination with a LED did not result in detectable temperature increases.

Results

TABLE 1
Ethanol produced (% v/v)/TIme

| LED | T3 | T4 | T5 |
|---|---|---|---|
| A (red) | 10.92 | 11.06 | 11.12 |
| B (darkness) | 10.06 | 10.42 | 10.8 |
| C (white) | 11.7 | 12.5 | 12.6 |
| D (yellow) | 1.9 | 2 | 1.191 |
| F (blue) | 10.72 | 11.81 | 11.7 |
| I (Infrared) | 14.5 | 14.2 | 13.24 |

Note:
T3 (36 hours), T4 (48 hours), T5 (60 hours)

TABLE 2
Ethanol Produced (only for T3)

| | | Concentration | Productivity |
|---|---|---|---|
| LED | % v/v | g/L | g/L/hour |
| A (red) | 10.92 | 85.2 | 2.37 |
| B (darkness) | 10.06 | 78.5 | 2.18 |
| C (white) | 11.7 | 91.3 | 2.54 |
| D (yellow) | 1.9 | 14.8 | 0.41 |
| F (blue) | 10.72 | 83.6 | 2.32 |
| I (Infrared) | 14.5 | 113.1 | 3.14 |

Note:
T3 is the minimum determined period of time when the best results are obtained.

These results show the best productivity (3.14 gram/liter/hour) for infrared light just after 36 hours of incubating. Fitting ethanol fermentation bioreactors with internal walls made of mirrors and, e.g., infrared LEDs, may result in great savings for the industrial production of Ethanol. However, it is possible that even better results may be obtained by cycling the light conditions. The method of the present invention allows for more experiments in which different cycling combinations can be used.

1. A method for optimizing ethanol fermentation, wherein said method comprises:
A. Inoculating yeast into culture medium in at least one container, wherein the culture medium has a fermentable carbon source, and wherein the container has completely transparent walls;
B. Putting the container inside a tridimensional closed system, wherein the tridimensional closed system has all internal surfaces with all the characteristics of a mirror;
C. Fitting the tridimensional closed system with a least one source of light, wherein the source of light is a light emitting diode (LED);
D. Incubating for at least one determined period of time;
wherein after incubating, ethanol is produced, and wherein the produced ethanol is separated from the culture medium.

2. The method of claim 1, wherein the yeast is a strain of Saccharomyces cereviseae.

3. The method of claim 1, wherein the fermentable carbon source is a saccharide starch.

4. The method of claim 1, wherein the fermentable carbon source is a saccharide sugar.

5. The method of claim 1, wherein all the tridimensional closed system internal surfaces are mirrors.

6. The method of claim 1, wherein the LED is selected from the group consisting of white light emitting LED, ultraviolet light emitting LED, violet light emitting LED, blue light emitting LED, green light emitting LED, yellow light emitting LED, orange light emitting LED, red light emitting LED, and infrared light emitting LED.

7. The method of claim 1, wherein said method comprises incubating the container inside de closed tridimensional system with a LED for a determined period of time, and incubating the container for an alternating period of time without any light.
